(19) United States
(12) Patent Application Publication
(10) Pub. No.: US 2007/0270597 A1
(43) Pub. Date: Nov. 22, 2007

(54) Process for preparing maleic anhydride in a microchannel reactor

(75) Inventors: Hagen Wilmer, Ludwigshafen (DE); Torsten Maurer, Frankenthal (DE); Frank Rosowski, Mannheim (DE); Rudolf Schuch, Schriesheim (DE)

Correspondence Address: CONNOLLY BOVE LODGE & HUTZ LLP, 1875 EYE STREET, N.W., SUITE 1100, WASHINGTON, DC, 20036, US

(73) Assignee: BASF Aktiengesellschaft, Ludwigshafen (DE)

(21) Appl. No.: 11/798,707

(22) Filed: May 16, 2007

(30) Foreign Application Priority Data
May 18, 2006 (EP) 06114165.1

(51) Int. Cl. C07D 307/34

(52) U.S. Cl. 549/259

(57) ABSTRACT

The present invention relates to a process for preparing maleic anhydride by heterogeneously catalyzed gas-phase oxidation of a hydrocarbon, which comprises feeding a hydrocarbon-comprising stream and a stream comprising oxygen or an oxygen source into a microchannel reactor and carrying out the reaction to form maleic anhydride in the explosive range in the microchannel reactor comprising the catalyst.

The present invention relates to a process for preparing maleic anhydride by heterogeneously catalyzed gas-phase oxidation of a hydrocarbon, which comprises feeding a hydrocarbon-comprising stream and a stream comprising oxygen or an oxygen source into a microchannel reactor and carrying out the reaction to form maleic anhydride in the explosive range in the microchannel reactor comprising the catalyst.

Maleic anhydride is an important intermediate in the synthesis of γ-butyrolactone, tetrahydrofuran and 1,4-butanediol, which are in turn used as solvents or are processed further to form, for example, polymers such as polytetrahydrofuran or polyvinylpyrrolidone.

The preparation of maleic anhydride by oxidation of hydrocarbons such as n-butane, n-butenes, benzene or propane as suitable catalysts has been known for a long time. This is generally carried out using vanadium-, phosphorus- and oxygen-comprising catalyst (cf. Ullmann's Encyclopedia of Industrial Chemistry, 6^{th} edition, 2000 electronic release, Chapter “MALEIC AND FUMARIC ACIDS, Maleic Anhydride-Production”), catalysts comprising vanadium and molybdenum mixed oxides (Tang et al., Appl. Catal. A 287 2005 197), or catalysts comprising molybdenum and vanadium mixed oxides (Nikolov V., Hungarian Journal of Industrial Chemistry (2000), 28(4); Bordes E., Topics in Catalysis (2000), 11/12(1-4); Zazhigalov V. A., Theoretical and Experimental Chemistry (Translation of Teoreticheskaya i Eksperimental'naya Khimiya) (2000) Volume Date 1999, 35(5); Guliants Vadim V., Catalysis Today (1999), 51(2); Cavani F., Trifiro F., Studies in Surface Science and Catalysis (1997), 110 (3rd World Congress on Oxidation Catalysis, 1997), 19-34; Cavani F., Cortelli C., Ligi S., Pierelli F.; Trifido F., DGMK Conference Report -/3 87-100 (2004); Guliants Vadim V., Carreon Moises A., Catalysis (2005), 18,1-45).

Since the oxidation of the hydrocarbons mentioned to form maleic anhydride is strongly exothermic, the reaction is generally carried out in a fixed-bed shell-and-tube reactor cooled by means of a salt bath. Depending on the size of the plant, this has from a few thousand to several tens of thousands of tubes charged with catalyst. The heat of reaction evolved is transferred via the wall of the tubes charged with catalyst to the surrounding salt bath, in general a eutectic mixture of potassium nitrate and sodium nitrate and potassium nitrite and sodium nitrite, and removed. Despite this salt bath cooling, a uniform temperature is not established over the length of the tubes charged with catalyst. Formation of overheated regions, known as hot spots, occurs. Thus, the hydrocarbon concentration of the reaction mixture in the vicinity of the point of entry into the tubes charged with catalyst is highest and that in the vicinity of the outlet is lowest, which leads to formation of the abovementioned superheated regions in the first half of the catalyst bed.

Furthermore, the excessive thermal stress influences the catalyst performance and the catalyst life. Since the reaction rate also increases with increasing temperature and thus results in ever more heat being produced, the formation of superheated regions can finally lead to an uncontrolled reaction, the consequence of which can be an “explosive runaway” reaction.

For economic and safety reasons, the shell-and-tube reactor is therefore operated so that the reaction temperature is as high as possible to ensure an economically attractive yield but the overheated regions established in the individual tubes do not lead to an uncontrolled reaction.

The preparation of maleic anhydride is accordingly restricted in terms of the process conditions for safety reasons. For example, explosive limits have to be taken into account when choosing the feed composition and maximum pressures have to be adhered to. When maleic anhydride is prepared in a fixed bed, n-butane concentrations are, for example, restricted to a maximum of 1.5-2.4% by volume and pressures up to a maximum of 3-5 bar are employed. The use of conventional reactors at higher pressures is not economical because the capital costs increase greatly with the pressure because of the need for explosion protection.

Approaches for increasing the yield are usually based on optimization of the process conditions by, for example, activity structuring of the catalyst bed by means of the geometry and/or doping or by temperature structuring of the catalyst bed or by recycling of the butane or the mixture of butane with air and/or water and/or by optimizing the catalysts used which are specifically matched to use in the respective reactor concept. Optimization of the catalysts can be effected, for example, by addition of doping metals or by means of specific methods of producing the catalyst or by addition of pore formers.

EP-A 593 646 describes a process for preparing maleic anhydride, in which the catalyst activity per unit volume of the bed varies with the temperature and the hydrocarbon concentration in the directional flow of the gas. The catalyst activity is set so that the reaction rate is promoted by a high activity in a region of low temperature and low hydrocarbon concentration within the bed and is restricted by a relatively low activity in a critical region within the bed where the combination of temperature and hydrogen concentration could otherwise result in the reaction proceeding at an excessive rate or cause an excessive increase in the gas temperature. In the examples, a yield of from 57 to 59% at 2.0 mol % of butane, a GHSV of from 1600 to 1650 h⁻¹ and a pressure of from 2 to 2.14 bar is achieved.

U.S. Pat. No. 5,168,090 describes a shaped oxidation catalyst structure for preparing maleic anhydride which comprises catalytic material comprising mixed oxides of vanadium and phosphorus and has (i) a geometric volume of from 30 percent to 67 percent of that of the void-free solid geometric shape, (ii) a ratio of external geometric surface area/geometric volume of at least 20 cm⁻¹, (iii) a bulk density of from 0.4 g/cm³ to 1.4 g/cm³ and (iv) sufficient mechanical strength. In the examples, a yield of 53% at a conversion of 87% is achieved at 1.5 mol % of butane, a pressure of 1.034 barg and a GHSV of 2000 h⁻¹.

EP-A 876 212 describes a phosphorus vanadium oxide catalyst for preparing maleic anhydride, wherein the catalyst comprises a shaped body having a volume of at least 0.01 cm³ and a BET surface area of at least 15 m²/g and said catalyst comprises molybdenum and has a molar ratio of molybdenum to vanadium of from 0.002 to 0.006, with molybdenum being concentrated essentially on the surface of the catalyst. In the examples, a maximum yield of 58.6 mol % is achieved at a maximum of 2.4% of butane, a space velocity of 2200 standard ml per gram of catalyst, 1.03 barg and a conversion of 85%.

EP-A 1 219 352 describes a process for preparing a phosphorus-vanadium oxide catalyst, in which a particulate pore modifier is used in the production of the catalyst precursor in ratios sufficient to achieve a concentration of the pore modifier of from 8 to 16% by weight and the precursor body is heated at a rate of from 1° C. to 3° C. per minute to a hold temperature which is not more than 15° C. below a threshold temperature. In the examples, a maximum yield of 61 mol % is achieved at 2.4% of butane, a GHSV of 1500/h, 1.034 barg and a conversion of 85%.

A further disadvantage of the use of shell-and-tube reactors is that, owing to the nonuniform temperature profile, the activation of the catalysts cannot take place in the shell-and-tube reactor but instead is typically carried out in an oven (EP-A 641 256) or belt calciner (WO 03/78310) before charging of the shell-and-tube reactors.

US 2004/0220434 describes the use of microchannel reactors in the conversion of n-butane into maleic anhydride for the first time. The reaction is carried out using a feed stream comprising n-butane and water in a volume ratio of 1:1 and air, with the air being mixed with the reactants in a volume ratio of 98:2. Consequently, the reaction is not carried out in the explosive range in US 2004/0220434. The explosive limit of n-butane is 1.4% at 20° C. and 1 bar of air.

In the doctoral thesis of Mr. Kah having the title “Entwicklung und Einsatz von Mikrostrukturreaktoren mit katalytisch wirksamen Strömungskanälen für die partielle Gasphasen-Oxidation von 1-Buten”, the use of microchannel reactors for the conversion of 1-butene into maleic anhydride is researched. The reactions carried out were carried out at a total pressure of 100 kPa and an n-butene concentration of from 0.45 to 5% by volume in air or 5% by volume in oxygen in the temperature range from 355 to 445° C. A maximum selectivity to maleic anhydride of 33% at a conversion of 83% was achieved in the microstructured reactors. No advantageous behavior of the reaction in the explosive range was able to be found in the experiments chosen (explosive limits of 1-butene in air according to GESTIS Databank: 1.2 (LEL)—10.6 (UEL) % by volume in air).

Despite the comprehensive prior art in the field of catalyst research, there is a continual need for optimization in respect of an improved yield at high capacity, in particular at the currently rising butane prices. If was accordingly an object of the present invention to provide a process for preparing maleic anhydride which gives higher yields compared to the prior art at-a high capacity. A further object was to provide a reactor in which both the activation of the catalysts and also the reaction to form maleic anhydride can be carried out.

It has been found that a process for preparing maleic anhydride by heterogeneously catalyzed gas-phase oxidation of a hydrocarbon, which comprises feeding a hydrocarbon-comprising stream and a stream comprising oxygen or an oxygen source into a microchannel reactor and carrying out the reaction to form maleic anhydride in the explosive range in the microchannel reactor comprising the catalyst, gives an above-average yield of maleic anhydride.

As hydrocarbons to be used in the process of the invention, it is advantageous to use aliphatic and aromatic, saturated and unsaturated hydrocarbons having at least three carbon atoms, for example propane, 1,3-butadiene, 1-butene, cis-2-butene, trans-2-butene, n-butane, C₄-mixture, 1,3-pentadiene, 1,4-pentadiene, 1-pentene, cis-2-pentene, trans-2-pentene, n-pentane, cyclopentadiene, dicyclopentadiene, cyclopentene, cyclopentane, C₅-mixture, hexenes, hexanes, cyclohexane and benzene. Preference is given to using propane, 1-butene, cis-2-butene, trans-2-butene, n-butane, benzene or mixtures thereof, in particular propane, n-butane or benzene. Particular preference is given to using n-butane, for example as pure n-butane or as a component of n-butane-comprising gases and liquids. The n-butane used can, for example, originate from natural gas, from steam crackers or FCC plants.

The addition of the hydrocarbon is generally carried out in a quantity-regulated manner, i.e. with continual specification of a defined amount per unit time. The hydrocarbon can be metered in liquid or gaseous form. It is preferably metered in liquid form with subsequent evaporation before entry into the shell-and-tube reactor unit.

As oxidant, use is made of oxygen-comprising gases such as air, synthetic air, an oxygen-enriched gas or “pure” oxygen, e.g. oxygen from the fractionation of air. The oxygen-comprising gas, too, is added in a quantity-regulated manner.

The process of the invention is carried out at a temperature of from 250 to 500° C. The temperature specified is, regardless of the type of reactor, in each case the mean temperature of the heat transfer medium. When n-butane is used as hydrocarbon starting material, the process of the invention is preferably carried out at a temperature of from 380 to 460° C. and particularly preferably from 380 to 440° C. When propane is used, the process of the invention is preferably carried out at from 250 to 350° C. When benzene is used, the process of the invention is preferably carried out at from 330 to 450° C.

The process of the invention is advantageously carried out isothermally, with a temperature which increases over the length of the reactor or with a combination of a temperature which increases over the length of the reactor and isothermal operation.

The process of the invention is advantageously carried out at an oxygen partial pressure of from 0.6 bar to 50 bar, preferably from 2 bar to 50 bar, particularly preferably from 3 bar to 50 bar, in particular from 4 bar to 50 bar.

The oxygen partial pressure, which is above that in the prior art, can advantageously be achieved either by increasing the total pressure to from 3 bar to 50 bar, preferably from 5 bar to 50 bar, in particular from 10 bar to 50 bar, or by introducing an increased amount of oxygen of from 20% by volume to 98% by volume, preferably from 40% by volume to 98% by volume, in particular from 60% by volume to 98% by volume, or by a combination of increasing the total pressure and increasing the amount of oxygen introduced.

In the process of the invention, the ratio of oxygen to hydrocarbon in the feed stream is advantageously from 10 to 50, preferably from 20 to 50, in particular from 30 to 50.

The n-butane concentration of the feed stream fed to the reactor unit is from 0.5 to 10% by volume, preferably from 0.8 to 10% by volume, particularly preferably from 1 to 10% by volume and very particularly preferably from 2 to 10% by volume.

A high n-butane concentration of from 2 to 10% by volume, preferably from 2.5 to 10% by volume, is preferred, particularly at high ratios of oxygen to butane of advantageously greater than 10, preferably greater than 20.

The n-butane conversion per pass through the reactor is from 40 to 100%, preferably from 50 to 95%, particularly preferably from 70 to 95% and in particular from 85 to 95%, of the n-butane in the stream at the inlet.

In the process of the invention, a GHSV (gas hourly space velocity) of preferably from 2000 to 10 000 h⁻¹ and particularly preferably from 3000 to 8000 h⁻¹, based on the volume of the stream fed at the inlet standardized to 0° C. and 0.1013 MPa abs and based on the reaction volume which is filled with catalyst or whose geometric surface is coated, is set in the reactor unit via the amount of the stream at the inlet.

The process of the invention can be carried out in two preferred process variants, viz. the variant with a “single pass” and the variant with “recirculation”. In the case of a “single pass”, maleic anhydride and, if appropriate, oxygenated hydrocarbon by-products are removed from the output from the reactor and the remaining gas mixture is discharged and, if appropriate, utilized thermally. In the case of “recirculation”, maleic anhydride and, if appropriate, oxygenated hydrocarbon by-products are likewise removed from the output from the reactor but part or all of the remaining gas mixture, which comprises unreacted hydrocarbon, is recirculated to the reactor. A further variant of “recirculation” is the removal of unreacted hydrocarbon and recirculation of this to the reactor.

The reaction products or the product stream can, if appropriate, be diluted at the end of the reactor or at the reactor outlet by addition of substances which are inert under the respective reaction conditions, for example water or nitrogen, so that a nonexplosive product stream is obtained. A nonexplosive product stream can also advantageously be obtained by means of a pressure increase. This product stream can then be worked up using conventional work-up units.

When using n-butane, a volatile phosphorus compound is advantageously added to the gas in the process of the invention to ensure a long catalyst operating life and to achieve a further increase in conversion, selectivity, yield, space velocity of the catalyst and space-time yield. Its concentration at the beginning, i.e. at the reactor inlet, is from 0.2 to 20 ppm by volume of the volatile phosphorus compounds based on the total volume of gas at the reactor inlet. A content of from 0.5 to 5 ppm by volume is preferred. For the purposes of the present invention, volatile phosphorus compounds are all phosphorus-comprising compounds which are in the gaseous state at the desired concentration under the use conditions. Preference is given to using triethyl phosphate or trimethyl phosphate as volatile phosphorus compound.

Generally known microchannel reactors are suitable for carrying out the process of the invention. In contrast to conventional reaction apparatuses, e.g. tube/shell-and-tube or fluidized-bed reactors, microchannel reactors offer, owing to the very small dimensions of the reaction channels (dimension in at least one spatial direction of <3 mm, preferably about 1 mm and less), inherent safety, i.e. propagation of flames or explosions is not possible (the diameter is below the minimal quench diameter). In terms of the way in which the process is carried out, there is increased freedom in terms of the choice of the organic/oxygen or air ratio, since explosion limits within the reactor do not have to be taken into account or adhered to. Design of the reactor for maximum explosion pressures is not necessary. Furthermore, short diffusion paths within the microstructures lead to greatly improved mass transfers and heat transfers which can be many times greater than those of conventional reaction apparatuses. Transport limitations which frequently occur in conventional shell-and-tube reactors are accordingly largely absent. Furthermore, the high heat removal potential of microchannel reactors makes more precise temperature control possible, so that, for example, the formation of hot spots can be suppressed and operation with an optimally selected axial temperature profile can be made possible. A runaway reaction in the reactor is effectively prevented.

Comprehensive descriptions of the configuration of microchannel reactors which in terms of their basic structure are suitable for carrying out the process of the invention may be found, for example, in US 2006/0036106 A1 and also in WO 02/18042 A1, which are hereby incorporated by reference.

For the purposes of the present invention, microchannel reactors or microreactors are reactors in general whose characteristic dimensions of the reaction channels, i.e. the dimensions in at least one spatial direction, e.g. height or width or diameter, are in the range from a few microns to a few millimeters, preferably <3 mm.

In large-scale industrial applications, too, the characteristic dimensions of the reaction space are retained. The increase in capacity is achieved by numbering-up, so that costly and time-consuming scale-up is dispensed with. The size of a production plant is thus flexible and can be inexpensively matched to requirements.

To introduce the catalysts into the microchannel reactor, it is possible to use all methods known to those skilled in the art. A comprehensive description of the prior art relating to this may be found in WO 01/12312 A2 and the references cited therein. The catalyst can be present, for example, as a wall coating which is bound firmly to the wall of the microreactor (cf. WO 01/12312 A2, pages 1 and 2, and references therein), or be introduced in the form of crushed material or shaped bodies as a fixed bed into the channels of the microreactor (cf. Tonkovich et al. (reference in WO 01/12312 A2, page 2)). Furthermore, the catalyst can be present as an insert, for example in the form of a metal foil or a metal mesh or metal gauze which is advantageously provided with a surface which is advantageous for the catalytic properties (e.g. a metal oxide surface). The active component is advantageously applied to or fixed on the surface (cf. WO 01/12312 A2, in particular pages 6 and 7, brief descriptions of the drawings).

In the case of a fixed bed, the catalyst which can be used in the process of the invention advantageously comprises shaped bodies having an essentially spherical geometry.

As catalysts in microchannel reactors, it is possible to use all catalysts which are generally suitable for the preparation of maleic anhydride, if appropriate with a suitable support material. Preference is given to using catalysts which are suitable for the conversion of n-butane, propane or benzene into maleic anhydride.

In the preparation of maleic anhydride from n-butane, it is advantageous to use vanadium-, phosphorus- and oxygen-comprising catalysts having a phosphorus/vanadium atomic ratio of from 0.9 to 1.5, preferably from 0.9 to 1.2, in particular from 1.0 to 1.1. The mean oxidation state of the vanadium is advantageously from +3.9 to +4.4 and preferably from 4.0 to 4.3. The catalysts used according to the invention advantageously have a BET surface area of >15 m²/g, preferably from >15 to 50 m²/g and in particular from >15 to 40 m²/g. They advantageously have a pore volume of >0.1 ml/g, preferably from 0.15 to 0.5 ml/g und in particular from 0.15 to 0.4 ml/g. The bulk density of the catalysts used according to the invention is advantageously from 0.5 to 1.5 kg/l and preferably from 0.5 to 1.0 kg/l.

The catalysts can comprise the vanadium-, phosphorus- and oxygen-comprising active composition in, for example, pure, undiluted form as “all-active catalyst” or diluted with a preferably oxidic support material as “mixed catalyst”. Suitable support materials for the mixed catalysts are, for example, aluminum oxide, silicon dioxide, aluminosilicates, zirconium dioxide, titanium dioxide or mixtures thereof. Preference is given to all-active catalysts.

The catalysts can further comprise additional promoters. Suitable promoters are the elements of groups 1 to 15 of the Periodic Table and their compounds. Suitable promoters are described, for example, in WO 97/12674 and WO 95/26817 and in U.S. Pat. No. 5,137,860, U.S. Pat. No. 5,296,436, U.S. Pat. No. 5,158,923 and U.S. Pat. No. 4,795,818. Preferred further promoters are compounds of the elements molybdenum, iron, zinc, hafnium, zirconium, titanium, chromium, manganese, nickel, copper, boron, silicon, tin, niobium, cobalt, lithium, antimony and bismuth, in particular molybdenum, iron, zinc, bismuth. The total content of promoters in the finished catalyst is generally not more than about 5% by weight, in each case calculated as oxide.

The catalysts can also comprise auxiliaries such as tableting aids or pore formers.

Furthermore, heteropolyacids known to those skilled in the art can also be used as catalytically active composition. Selectivities to maleic anhydride of 90% are usually achieved at n-butane conversions of 15% and selectivities of 46% to maleic anhydride are usually achieved at conversions of 62% (Davis et al., Angew. Chem. (2002) 114, 886-888; Holles et al., J. Catal. (2003) 218, 42-66).

The catalysts mentioned can be produced by all methods known to those skilled in the art. The catalysts used according to the invention can, for example, be produced as described in the patents U.S. Pat. No. 5,275,996 and U.S. Pat. No. 5,641,722 or the published specification WO 97/12674. Shaping is preferably effected by tableting.

In the prior art, the production of the catalyst is generally described as a multistage process in which a catalyst precursor is firstly produced and this is subsequently converted into the active form by calcination. The catalyst precursors which can be used in the process of the invention can be produced, for example, as described in the documents U.S. Pat. No. 5,275,996, U.S. Pat. No. 5,641,722, WO 97/12674, WO 01/68626, WO 01/68245, WO 02/22257, WO 02/34387, DE 102 11 449 A1, DE 102 11 445 A1, DE 102 11 447 A1, DE 102 11 446 A1 and DE 102 35 355 A1.

In the process of the invention, the activation can, in contrast to the prior art, take place directly in the microchannel reactor.

In the preparation of maleic anhydride from propane, it is advantageous to use catalysts based on vanadium and molybdenum mixed oxides (Tang et al., Appl. Catal. A 287 2005 197). The V/Mo ratio advantageously ranges from 1/9 to 3/7, preferably from 1/4 to 3/7. The active composition can advantageously comprise doping components in addition to the two main components vanadium and molybdenum in order to increase the activity or selectivity of the catalyst. Ag, Cu and Zn are particularly preferably used as doping components. The production of the catalyst is carried out by methods known to those skilled in the art, which are described, for example, in Tang et al., Appl. Catal. A 287 2005 197.

In the preparation of maleic anhydride from benzene, it is advantageous to use coated catalysts having supported active compositions based on molybdenum and vanadium mixed oxides. The MoN ratio advantageously ranges from 1/2.5 to 1/5. The proportion of active composition in the coated catalyst is advantageously from 10 to 20% by weight. The active composition can advantageously comprise doping components in addition to the two main components molybdenum and vanadium in order to increase the activity or selectivity of the catalyst. Advantageous doping components are Ag, Na (Bielanski et al., Bull. Acad. Pol. Sci., Ser. Sci. Chim. (1976) 24(5), 415-23), rare earths such as, for example, Tb, Dy, Gd or Er (Khiteeva et al., Zh. Fiz. Kihm. (1981) 55(8), 2121-2), Cr, Co (Bielanski et al., Bull. Acad. Pol. Sci. Ser. Sci. Chim. (1976) 24(6), 485-92, Zn, Cu (Ionita et al., Rev. Chim. (Bucharest) (1968) 19(2), 105-7). Other promoters which are used in the oxidation of o-xylene to phthalic anhydride, e.g. Cs and P, can also be used advantageously. The production of the catalyst is carried out by methods known to those skilled in the art, which are described, for example, in Ullmann's Encyclopedia of Industrial Chemistry, 6^{th} edition, 2000 electronic release, Chapter 2.2.1.

The process of the invention gives a yield which is improved by 10% compared to the prior art. Furthermore, the separate process step of activation can be saved, since activation can be carried out directly in the microchannel reactor.

1. A process for preparing maleic anhydride by heterogeneously catalyzed gas-phase oxidation of a hydrocarbon, comprising feeding a hydrocarbon-comprising stream and a stream comprising oxygen or an oxygen source into a microchannel reactor and forming maleic anhydride in the explosive range in the microchannel reactor wherein the microchannel reactor comprises a catalyst.

2. The process according to claim 1, wherein the reaction forming maleic anhydride is conducted at an oxygen partial pressure of from 0.6 to 50 bar.

3. The process according to claim 1, wherein the reaction to forming maleic anhydride is conducted at a total pressure of from 3 to 50 bar.

4. The process according to claim 1, wherein the reaction forming maleic anhydride is conducted in the presence of from 20 to 98% by volume of oxygen.

5. The process according to claim 1, wherein the reaction forming maleic anhydride is conducted at a ratio of oxygen to hydrocarbon of from 10 to 50.

6. The process according to claim 1, wherein n-butane, propane or benzene is utilized as the hydrocarbon.

7. The process according to claim 1, wherein n-butane is utilized as the hydrocarbon and a vanadium-, phosphorus- and oxygen-comprising catalyst having a phosphorus/vanadium atomic ratio of from 0.9 to 1.5 is utilized as the catalyst.

8. The process according to claim 1, wherein a catalyst precursor is activated in the microchannel reactor.

9. The process according to claim 2, wherein the reaction forming maleic anhydride is conducted at a total pressure of from 3 to 50 bar.

10. The process according to claim 2, wherein the reaction forming maleic anhydride is conducted in the presence of from 20 to 98% by volume of oxygen.

11. The process according to claim 3, wherein the reaction forming maleic anhydride is conducted in the presence of from 20 to 98% by volume of oxygen.

12. The process according to claim 2, wherein the reaction forming maleic anhydride is conducted at a ratio of oxygen to hydrocarbon of from 10 to 50.

13. The process according to claim 3, wherein the reaction forming maleic anhydride is conducted at a ratio of oxygen to hydrocarbon of from 10 to 50.

14. The process according to claim 4, wherein the reaction forming maleic anhydride is conducted at a ratio of oxygen to hydrocarbon of from 10 to 50.

15. The process according to claim 2, wherein n-butane, propane or benzene is utilized as the hydrocarbon.

16. The process according to claim 3, wherein n-butane, propane or benzene is utilized as the hydrocarbon.

17. The process according to claim 4, wherein n-butane, propane or benzene is utilized as the hydrocarbon.

18. The process according to claim 5, wherein n-butane, propane or benzene is utilized as the hydrocarbon.

19. The process according to claim 2, wherein n-butane is utilized as the hydrocarbon and a vanadium-, phosphorus- and oxygen-comprising catalyst having a phosphorus/vanadium atomic ratio of from 0.9 to 1.5 is utilized as the catalyst.

20. The process according to claim 3, wherein n-butane is utilized as the hydrocarbon and a vanadium-, phosphorus- and oxygen-comprising catalyst having a phosphorus/vanadium atomic ratio of from 0.9 to 1.5 is utilized as the catalyst.
